(19) United States
(12) Patent Application Publication
(10) Pub. No.: US 2005/0059814 A1
(43) Pub. Date: Mar. 17, 2005

(54) Alkylated c-sugars and uses thereof

(76) Inventors: Arno F. Spatola, Louisville, KY (US); Jackie B. Spatola, Louisville, KY (US); Grant K. Taylor, Louisville, KY (US); Florence Brunel, San Diego, CA (US)

Correspondence Address: David W Carrithers;Carrithers Law Office, One Paragon Centre, 6060 Dutchman's Lane Suite 140, Louisville, KY, 40205, US

(21) Appl. No.: 10/471,328

(22) PCT Filed: Mar. 11, 2002
(86) PCT No.: PCT/US02/07092

Related U.S. Application Data
(60) Provisional application No. 60/274,860, filed on Mar. 9, 2001.

(51) Int. Cl. C07H 15/04; C07H 5/06

(52) U.S. Cl. 536/18.7; 536/120

(57) ABSTRACT

The present invention relates to alkylated C-sugars which are new carbohydrate derivatives based on the “C-Sugar” platform. These alkylated C-sugars are converted from hydrophilic, hydrogen-bonded saccaride derivatives and are very stable, highly soluble and relatively low molecular weight structures. These alkylated C-sugars can serve as effective bioconjugates and pharmaceutical carriers. The alkylated C-sugar have the formula wherein n=0, 1, 2, 3, 4, Rₓ=aryl, alkyl, or halogen-substituted aryl or alkyl, Y=(CH₂)ₘNH₂,(CH₂)ₘCO₂H,(CH₂)ₘOH,(CH₂)ₘCH, or (CH₂)ₘCl, and m=0, 1, 2 or 3.

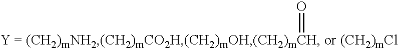

This application is a U.S. non-provisional application which claims the benefit of U.S. provisional application Ser. No. 60/274,860 filed Mar. 9, 2001, the teachings of which are hereby incorporated by reference.

### BACKGROUND OF THE INVENTION

1. Field of the Invention

The present invention relates to alkylated C-sugars which are new carbohydrate derivatives based on the “C-Sugar” platform. These alkylated C-sugars are converted from hydrophilic, hydrogen-bonded saccharide derivatives and are very stable, highly soluble and relatively low molecular weight structures. These alkylated C-sugars can serve as effective bioconjugates and pharmaceutical carriers.

2. Description of the Related Art

Advances in the synthetic chemistry of oligosaccharides, the most structurally diverse biopolymers, have been driven principally by the growing understanding of their role as encoders of biological information. Glycoconjugates on cell surfaces have a major role in biological phenomena such as the immune response, intercellular recognition, cellular adhesion, intracellular targeting, cell growth regulation, metastasis, and inflammation (Dwek, 1996; Kahane, 1996; Garegg, 1993). Glycosylation of proteins affects a variety of properties such as folding, packing, proteolytic resistance, conformational stability, quaternary structure, and water structure (Dwek, 1996; St. Hilaire, 1998).

For these reasons, interest is strong in the creation and biological evaluation of oligosaccharide and glycopeptide libraries (Armstrong, 1996; Seeberger, 1996; Sofia, 1998). While cell surface carbohydrates are, typically, oligosaccharides, recognition by a protein typically involves a monosaccharide or disaccharide unit of the oligosaccharide. Consequently, monoglycosylated amino acids (and analogs) and a few glycosyl-glycosylated amino acids have been the building blocks of glycopeptide libraries. More interest has focused on the glycopeptide libraries, principally because cell surface carbohydrates, while they bind to proteins with high specificity, typically have weak binding constants (and limited bioavailability due to their biological instability). With glycopeptide libraries, the principal aim has been to create glycomimetics targeted for lectins, enzymes, and other cell surface receptors which bind carbohydrates (St. Hilaire et al., 1998; Sutherlin et al., 1996). For example, considerable interest has focused on E-selectin and its ligand, the sialyl Lewis X antigen because of their role in inflammation. Glycomimetics of sialyl Lewis X, a tetrasaccharide, have been found with binding potencies higher than the natural compound (Bertozzi, 1992) and glycosylation of peptides is known to enhance binding to this receptor (St. Hilaire, 1998).

Various methods are available for developing small molecule therapeutics ranging from structure-based combinatorial techniques (Ellman, 1996; Gallop, 1994), computer screening approaches (Li et al., 1997), and various peptidomimetic strategies (Hruby, 1993; Spatola, 1983; Sawyer, 1997). Once a lead is identified, the developmental emphasis is directed toward optimizing potency, enhancing receptor or target specificity, and ensuring bioavailability with growing emphasis on nuclear-based therapies. It is clear that entry into the nucleus through membrane barriers becomes a formal obstacle. Various workers have reported success with pro-drugs, other bioconjugates (Niidome, 1999; Prouillat, 1998), and tactics designed to mask difficulties such as desolvation penalties by using, for example, N-methyl amides to enhance the solubility of polyamide species such as peptides (Conradi, 1992). More recently, there has been renewed interest in stabilized glycopeptides as drug conjugates.

Currently, polyethylene glycol (PEG) is used as an effective bioconjugate because of its global solubility, enhanced stability through the ester linkage, and an optimum blend of non-reactivity and non-immunogenicity (Greenwald, 2000, Zalipsky, 1995). This knowledge and the benefits of glycoconjugation has led the present inventors to research C-sugars as bioconjugates and determine their effect when coupled to peptides and drugs. Through their research, the present inventors believe that the alkylated C-sugars of the present invention are particularly effective as bioconjugates. The presently claimed alkylated C-sugars possess some of the beneficial attributes of PEG and additionally provides stereochemical versatility via five stereocenters. The alkylated C-sugars can be easily and covalently linked to either a backbone or side chain peptide or pseudopeptide functionality or a drug. Thus, the invention has been completed based on this understanding and research.

### SUMMARY OF THE INVENTION

The present invention is directed to alkylated C-sugars of the following formula: 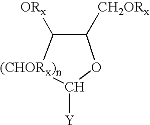
wherein n=0, 1, 2, 3 or 4,
   Rₓ=aryl, alkyl, or halogen-substituted aryl or alkyl,

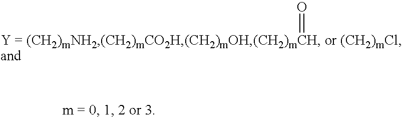

The present invention is also directed to compositions comprising a drug and the alkylated C-sugars, as well as methods of making and using said alkylated C-sugars and compositions.

### DESCRIPTION OF THE PREFERRED EMBODIMENT

C-sugars are derivatives of carbohydrates in which the anomeric carbon is replaced by a methylene unit. The presently claimed alkylated C-sugars utilizes C-sugars instead of carbohydrates or polymers as the core conjugate. Through alkylation, the inventors have eliminated hydroxyl groups in favor of ether linkages.

As background, the methods for making the alkylated C-sugars of the present invention starts with the basic approaches used in the prior art. As an example, the stereoselective synthesis of alpha and beta C-glucosides in Scheme 1 (see below) is representative of an approach used in the prior art (Lewis, 1982). The approach to Sa and 6a of Scheme 2 works equally well for the synthesis of alpha-mannoside 7a, and this is readily converted to 7b in high yield (Tang, 1998). The beta-linked C-glycoside 8a (Scheme 2) was obtained by Bertozzi employing free radical chemistry and the subtle manipulation of phthalimido N-protecting groups (Roe, 1996), while 8b was obtain by Hollingsworth using direct SN2 alkylation of a halo sugar (Kim, 1994). These methods should be adaptable to the synthesis of 9a and 9b (Scheme 2) as well. Compounds 7, 8, and 9 can be the starting points for the construction of an initial set of C-glycosylated amino acid building blocks which is an embodiment of the present invention.

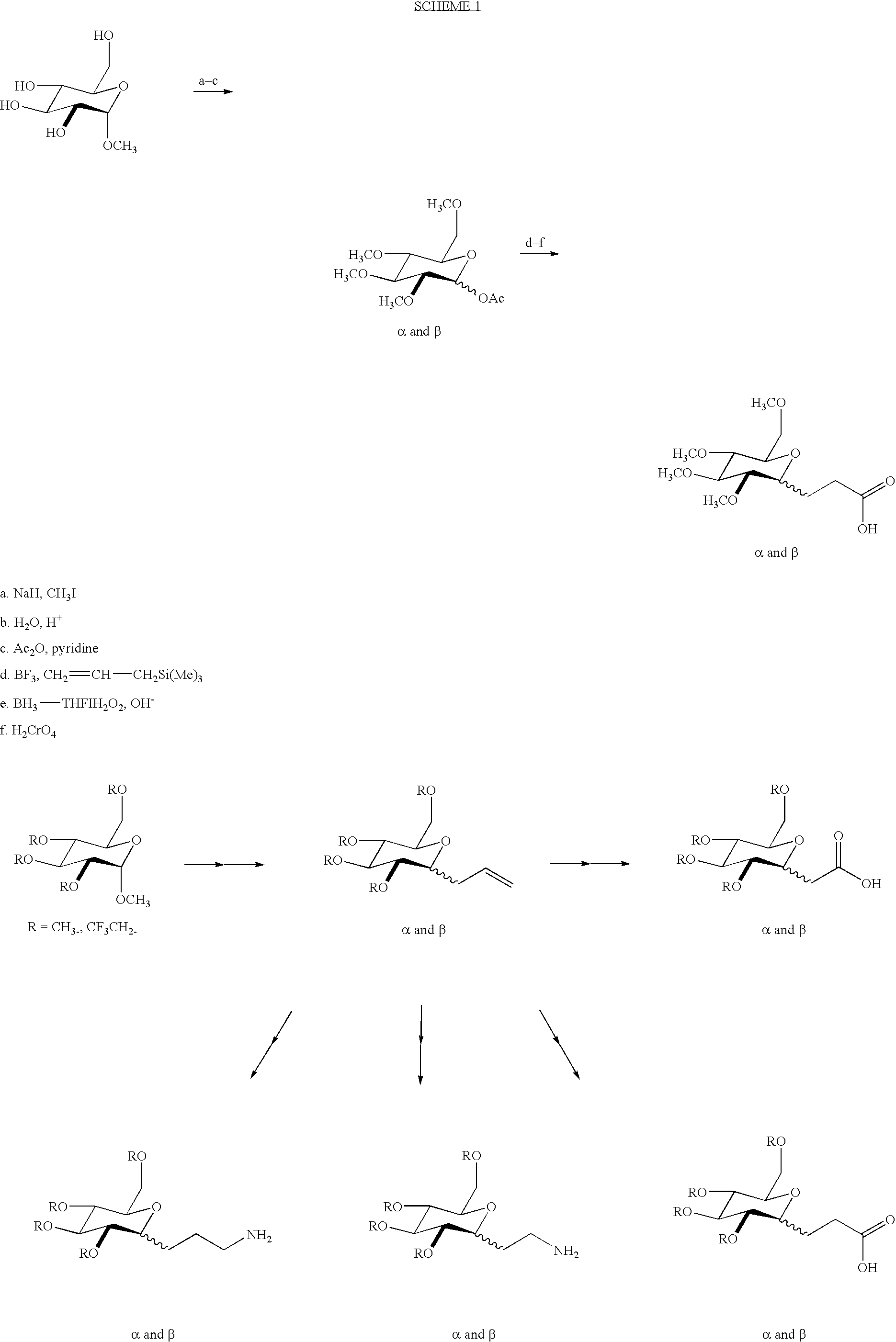

The key step in the synthesis of beta-GlcNAc—CH₂CO—NH-Lys (Scheme 2) is the carbodiimide-mediated coupling of protected GlcNAc derivative 10 with lysine derivative 11 (P=Boc or Fmoc). While Hollingsworth and Bertozzi used acetyl protection of OH groups for their synthesis of 8, either route is adaptable employing benzyl ether protection. Benzyl ether protection has been employed in the synthesis of the analogous GlcNAc—C-glycoside 12 (Liang, 1996). Benzyl ether protection is preferable to acetyl in this case because it offers orthogonality of conditions for deprotection and enhanced stability for solid phase synthesis of oligopeptides. Further, if HF deprotection conditions result in the Friedel Crafts alkylation of Tyr residues that might be present in the glycopeptides, then 2,6-dichloro-benzyl ether protection could be employed. For the synthesis of alpha-Man-CH₂CO—NH-Lys, alpha-Man-CH₂CO—NH-Lys can be prepared by an analogous coupling of benzyl ether-protected 7b (R═H) (Tang, 1998). Both D and L amino acids would be used affording both diastereoisomers for use in glycopeptide assembly.

The basic approach for the synthesis of the alpha and beta-linked C-serine analogs can be illustrated by reference to the case of alpha-Man-CH₂Ser. Scheme 2 outlines the synthesis of alpha-Man-CH₂Ser. Those skilled in the art can follow the teachings of Scheme 2 below to make any desired C-Sugar.

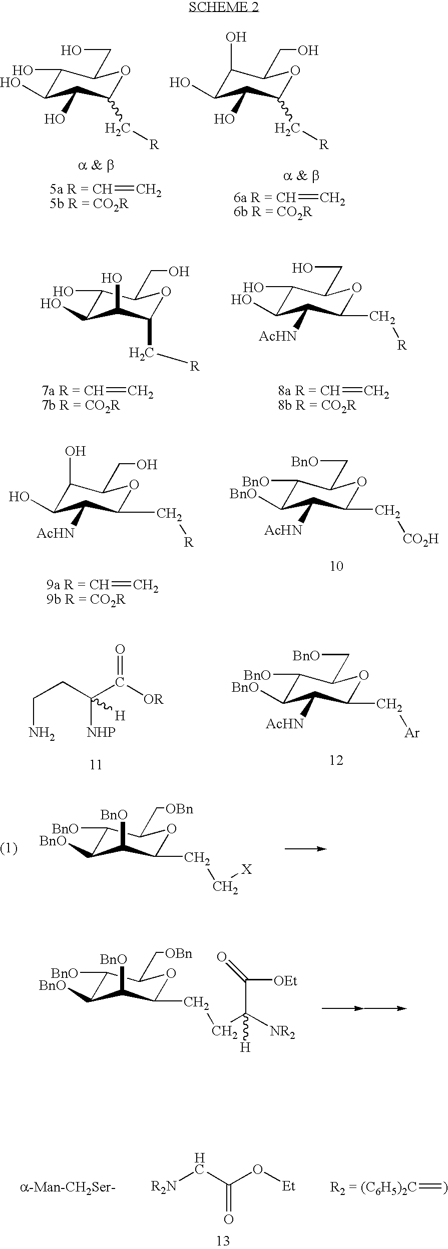

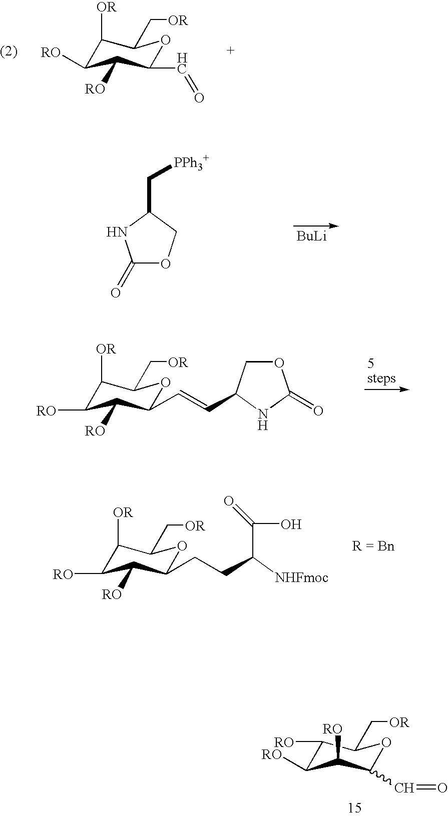

The ester group of 7b (or the aldehyde group from the ozonization of 7a) will be reduced and the resulting alcohol converted to a sulfonate or some other leaving group. A glycine anion 13 (Luxen, 1993) can be used for amino acid synthesis and it can be employed as shown in Scheme 2, Reaction (1) above. The reaction will give a mixture of L and D diastereoisomers which is what is desired since both will be employed in glycopeptide formation. The diastereoisomers can be separated by chromatography. In the case of the GlcNAc— and GalNAc—CH₂Ser derivatives, phthalimido protection may have to be employed for N in the event of H abstraction by the nucleophile or base.

An alternative approach to C-serine derivatives has been developed by Bertozzi (Bertozzi, 1992). This approach is summarized as Reaction (2) of Scheme 2 above. One skilled in the art should be familiar with the synthesis of Cl aldehyde derivatives such as 14. For example, the alpha and beta mannose derivative 15 has been the starting point for a number of C-mannosides (Zhang, 1998). An advantage of the approach of reaction pathway (2) is that it allows for the selection of the amino acid stereoisomer, either L or D. The disadvantage is that isomerization of an aldehyde a isomer to the more thermodynamically stable b isomer is facile, often occurring under conditions of reaction at the aldehyde center. Consequently, this pathway appears to be limited to the synthesis of only b-linked C-glycosyl derivatives. Nonetheless, it remains an alternative approach that has been demonstrated to work.

### Scheme 3

The following scheme teaches the synthesis of an alkylated mannosylacetic acid which is a specific embodiment of the present invention:

### Synthesis of Permethylated Mannosylacetic Acid

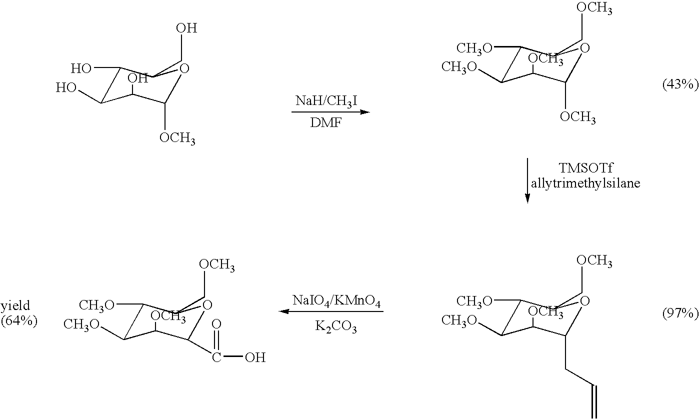 [0021] a. Allylation A. Hosami, Y. Sakata, H. Sakurai, “Highly Stereoselective C-allylation of Glycopyranosides with Allylsilanes Catalyzed by Silyltriflate or Iodosilane,” Tetrahedron Lett., 1984, 25, 2383.
b. Oxidation P. A. Aristoff, P. D. Johnson, and A. W. Harrison, “Total Synthesis of a Novel Antiulcer Agent via a Modification of the Intramolecular Wadsworth-Emmons-Wittig Reaction,” JACS, 1985, 107, 7967-7974.

As described earlier, the C-sugars are typically prepared in an O-benzyl protected form. The derivatives can then be coupled to a peptide-free amine using standard condensation procedures and the remaining peptide and carbohydrate protecting groups are then removed, usually using catalytic hydrogenation. This leaves a hydrophilic compound that can increase aqueous solubility and balance excessive peptide hydrophobic character.

Analogously, the alkylated C-sugars of the present invention can thus be readily prepared as, for example, an O-methyl rather than O-benzyl derivative. But following peptide condensation, the O-methylation will be retained. These ether compounds should now have solubility properties comparable to polyethylene glycol (PEG), the well-known bioconjugate.

After synthesis of a protected C-glycosyl residue or its desired amino acid derivative, conditions for its use in solid phase peptide construction will be optimized. This will be a necessary step, one which can be performed based on the teachings of others (Bertozzi, 1992; St. Hilaire, 1998; Kutterer, 1999).

Once the alkylated C-sugars are prepared, appropriate coupling reagents should be selected. The alkylated C-sugars, with, for example, a free carboxylic acid residue, will be condensed to the N-terminus of the appropriate linear peptides or a pseudopeptide using traditional peptide condensing agents. Deprotection and cleavage from the solid support will be accomplished with anhydrous hydrogen fluoride (HF) (Sakakibara, 1971) singly or in groups (Houghten, 1986). Alternatively, if HF proves unusually harsh for the carbohydrate component, inventors advise utilizing a two step cleavage and deprotection approach using two methods previously developed: phase transfer catalysis for peptide-solid phase resin cleavage (Anwer and Spatola, 1992) or by ammonium formate catalytic transfer hydrogenolysis (Anwer et al., 1983). Alternatively, traditional hydrogenation at elevated temperature and pressure can be used if any of the hydroxylbenzyl functions prove refractory to the standard deprotection methods.

As an additional example of a functionalized alkylated C-sugar, a simplified method of synthesis for an embodiment of the present invention can be set forth as follows in Scheme 4:

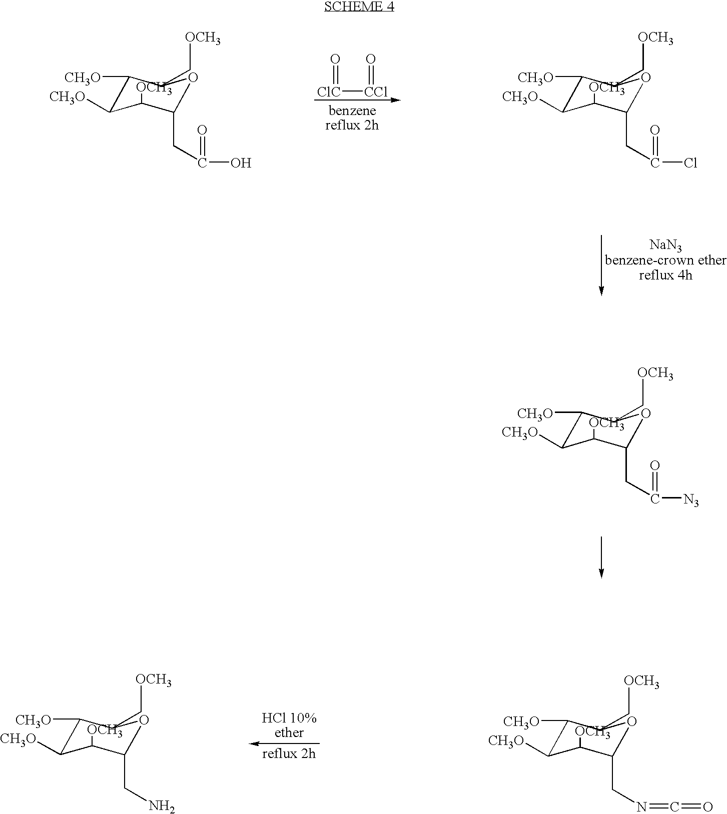

Thus, the alkylated C-sugars of the present invention can be structured to comprise a free carboxylic acid, amine group or any other appropriate functional group which facilitates the coupling of the C-sugar bioconjugate to a drug or peptide. Also, the C-sugars may thus be coupled to a wide variety of drugs and/or peptides for the purpose of improving their solubility, membrane transport, and overall bioavailability. Such variability in the types of coupling partners is shown in Scheme 5 below:

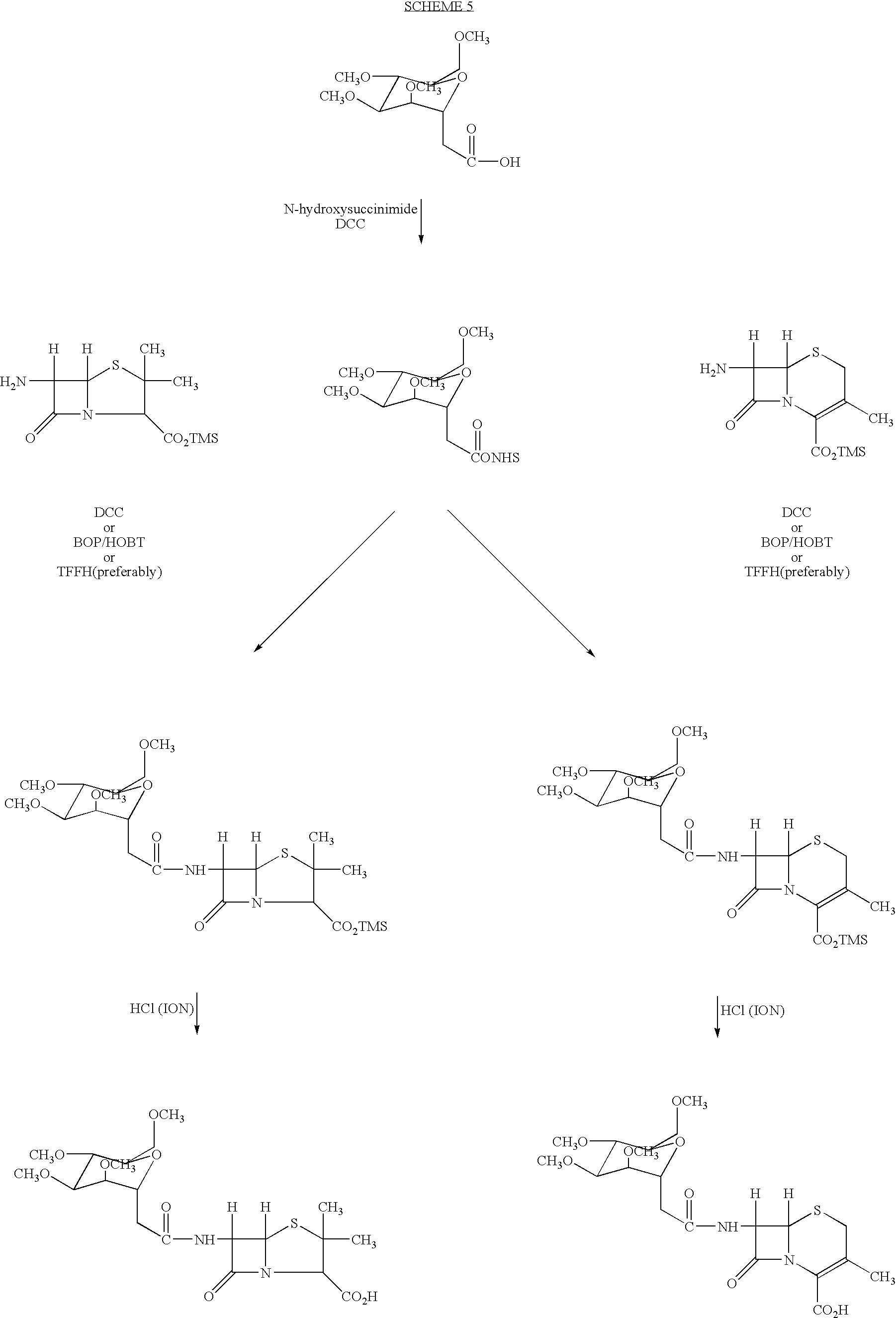

Among the other variables for the claimed alkylated C-sugars in addition to a free functional group are the ring size, nature, and stereochemistry of the parent carbohydrates (such as mannose, glucose, and galactose) and the nature of the O-alkyl derivatives (O-methyl: Rₓ═CH₃; O-ethyl: Rₓ═CH₂CH₃; trifluoromethyl, R═CF₃; D-2,2,2-trifluoroethyl, R═CH₂CF₃, as well as other alkyl, substituted alky, aryl and other heterocyclic variants).

Moreover, both linear and cyclic peptide drug candidates may be enhanced by the attachment of the bioconjugates of the present invention. Beneficial properties of cyclic peptides, are also set forth in our co-pending provisional application Ser. No. 60/274,846 entitled STABILIZED L-Xxx-Yyy-L-L PEPTIDOMIMETICS are further enhanced by the stabilization of the carbohydrate moiety, as is described herein.

It is important to note that although the above disclosure primarily teaches the synthesis of N-terminal and side chain modified glycopeptides, the alkylated C-sugars taught herein can be linked to many desired molecules and as such, are considered to be novel bioconjugates. One skilled in the art can easily link the claimed alkylated C-sugars to many desired molecules based on the knowledge readily possessed and available to one skilled in the art. The desired molecules can be any substance (i.e. drug) which is intended for use in the diagnosis, cure, mitigation, treatment, or prevention of disease, and have an effect in the structure or function of the body so as to achieve its intended use. Thus, the present invention should not be limited to the synthesis of N-terminal, C-terminal and side chain modified glycopeptides disclosed above. Instead, the present invention should be interpreted to encompass any composition comprising a drug and the presently claimed alkylated C-Sugars.

As stated above, addition of a permethylated C-sugar to peptides can be conveniently performed through standard amide coupling reactions to the N-terminus, the C-terminus, or even to side chain functional groups. In the latter case, an acid functionalized C-sugar is attached to lysine while an amine substituted C-sugar may be coupled to side chain carboxylic acids such as those found in aspartic acid or glutamic acid. By selecting other functional group combinations, additional linkages to either linear or cyclic peptides may be effected using techniques well known to those skilled in the art.

In the present example, a C-sugar with a carboxylic acid group is added to the N-terminus of a cyclic peptide that has been prepared by solid phase methods of synthesis. The final product retains the major structural characteristics required for its biological action but now is modified by a bioconjugate that provides important solubility advantages. In a similar fashion, other drug candidates may be appropriately modified by one or more alkylated C-sugars to assist in the transport and bioavailability of the derivatized compound.

The present invention is further specifically described according to the following examples. However, the Examples are merely illustrative in nature and should not be construed to limit the scope of the claims.

### EXPERIMENTAL

Synthetic and Analytical Conditions:

KMnO₄, NaIO₄, a methyl D mannopyranose, CH₃I, NaH, allyltrimethylsilane, and TMSOTf were purchased from Aldrich, and Silica gel (60 Å 70-230/mesh) was purchased from Whatman.

Thin layer chromatography (TLC) was performed on Merck-254 silica plates in the following systems (V/V):
A. Ethyl acetate:hexane (1:1)
B. Ethyl acetate:hexane (1:2)

The Rf values are expressed with appropriate parenthetical abbreviations. The plates were sprayed with a mixture of H₂SO₄/HCl and heated to be visualized.

Purification was performed by column chromatography using silica gel.

Molecular weight determinations were made on a Matrix-Assisted-Laser Desorption Ionization-Time of flight (MALDI-TOF) Mass Spectrometer Model, DE-Pro, made by Perceptive Biosystems (now Applied Biosystems) as well as on a QUATTRO-LCZ, Electron-Spray Ionization Quadripole Mass Analyzer, made by Micromass.

### Example 1

### Synthesis of a Methylated D-Mannosylacetic Acid and Coupling to a Peptide

A. Synthesis of the Permethylated Mannoside

The 5.84 g (0.03 mole) of a methyl D mannopyranoside was dissolved in freshly distilled DMF in a 3 neck round bottom flask equipped with a condenser under a flow of argon. A two fold excess NaH (1.45 g, 0.06 moles) was added and the temperature was increased to 75° C. After two hours, the mixture was cooled and a two fold excess of CH₃I (3.76 ml, 0.06 mole) was added. After an hour, the process was repeated three to four times depending on the TLC of the mixture.

The mixture was then quenched by the dropwise addition of methanol and extracted with EtOAc and brine. The organic layer was dried over Na₂CO₃ and the solvent was removed by rotary evaporation. The oil was dried overnight under high vacuum and 5.21 g (69%) was obtained.

The compound was purified on a silica column with a solvent mixture of EtOAc:hexane (1:2) then (1:1). 3.2 g (43%) of pure product was obtained. The compound was characterized by TLC, ¹H and ¹³C NMR. R_{f}=0.4 in A.

B. Allylation of the Permethylated Mannoside

The 2.82 g (0.011 mole) of permethylated mannoside was dissolved in CH₃CN at 0° C. under argon. The allyltrimethylsilane 3.5 ml (0.022 moles) was added at once and then 2.03 ml (0.011 mole) TMSOTf was slowly added. Completion of the reaction was checked after two hours. The reaction was quenched by the dropwise addition of methanol and extracted with EtOAc and brine. The organic layer was dried over Na₂CO₃ and the solvent was removed by rotary evaporation. The oil was dried overnight under high vacuum and a yellowish oil was obtained.

The compound was purified by column chromatography with a mixture of EtOAc:hexane (1:3), then (1:2). 2.86 g (97%) of pure compound was obtained. The alkene was characterized by TLC, 1H and ¹³C NMR. Rf=0.14 in system B.

C. Oxidation of the Alkene

Aristoff's procedure to transform an alkene into an acid was applied.

A solution of 20 g (93 mmol) of sodium metaperiodate in 300 ml of distilled water was treated with 0.33 g (2 mmol) of potassium permanganate, stirred 30 min at 25° C., and treated with 1.6 g (12 mmol) of anhydrous potassium carbonate, and then 2.8 g (11 mmol) of the alkene in 80 ml of tert-butanol was added.

The resulting reddish-purple suspension was stirred for 2 hours at 25° C., heated with 2 ml (30 mmol) of ethylene glycol, stirred for 2 hours, acidified to pH=2 with 1M aqueous hydrochloric acid, and extracted with chloroform. The combined organic extracts were washed with brine and dried (Na₂SO₄). The solvents were removed in vacuum to give 1.9 g of acid as a yellowish oil (64%).

The compound was characterized by ¹H, ¹³C NMR, IR, MALDI-TOF and ionization mass spectrometry. C₁₂H₂₂O₇ mass calculated: 278.29 mass found [M-1]: 277.3

D. Synthesis of an Alkylated C-Sugar Linked at the Amino Terminus of a Cyclic Peptide.

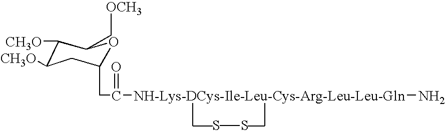

The compound: 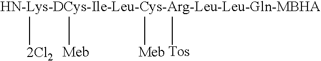
was assembled by classical methods using solid phase synthesis. 0.1 mmol of peptide loaded on the resin was treated with 0.2 mmol of permethylated mannosylacetic acid in freshly distilled DMF with TFFH (0.4 mmol) as a coupling agent and diisopropylethylamine as base.

After 2 hours the coupling was complete according to a Kaiser (ninhydrin) test. The resin was washed with CH₂Cl₂/EtOH/CH₂Cl₂ and treated with anhydrous HF for 1 hour under standard conditions. The compound obtained (88% yield) was checked by mass spectrometry and the expected structure was confirmed. The disulfide bridge was formed quantitatively overnight by mixing the peptide with DMSO (excess). The final mass of the compound was checked by mass spectrometry and confirmed.

Calculated for M+H: 1347.7 Found: 1347.7

### Example 2

### Synthesis of an Amine Funtionalized Methylated Mannose C-Sugar

Following the methods disclosed above and from knowledge commonly available and possessed by one skilled in the art as reflected in the incorporated references cited herein, an aminomethyl version of an alkylated C-sugar can be synthesized based on the scheme shown below:

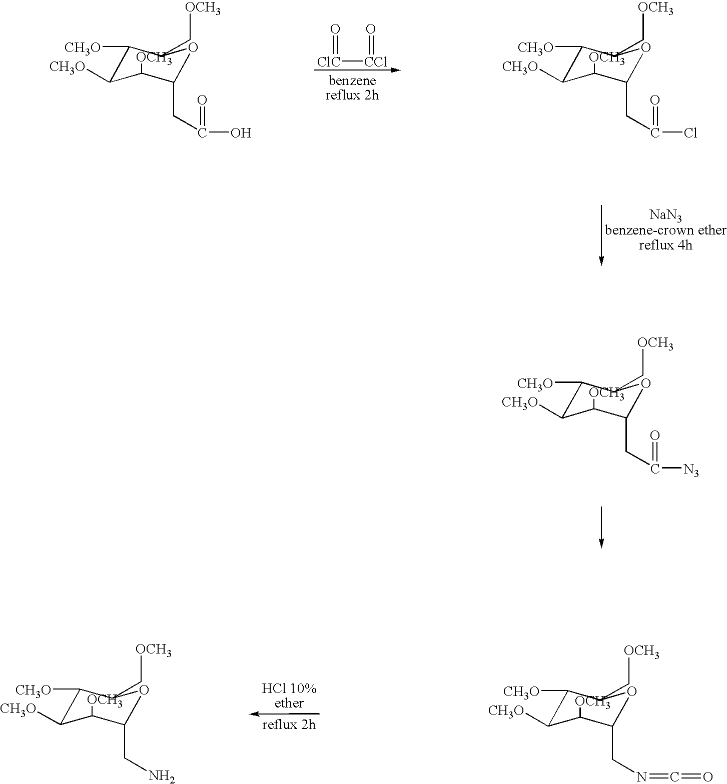

### Example 3

### Synthesis of Alkalated C-Sugar Derivatives of Penicillin and Cephalasporin

As stated above, the synthesis of N-terminal, C-terminal and side chain modified glycopeptides is only one of many embodiments of the present invention. One skilled in the art can also easily link the presently claimed alkylated C-sugars to many desired molecules such as a drug based on the knowledge readily possessed and available to one skilled in the art as reflected in the incorporated references cited herein.

As an example, following the methods disclosed above and from knowledge commonly available and possessed by one skilled in the art, a functionalized alkylated C-sugar can be attached to an amino-penicillinic and amino-cephalasporic acid based on the scheme shown below.

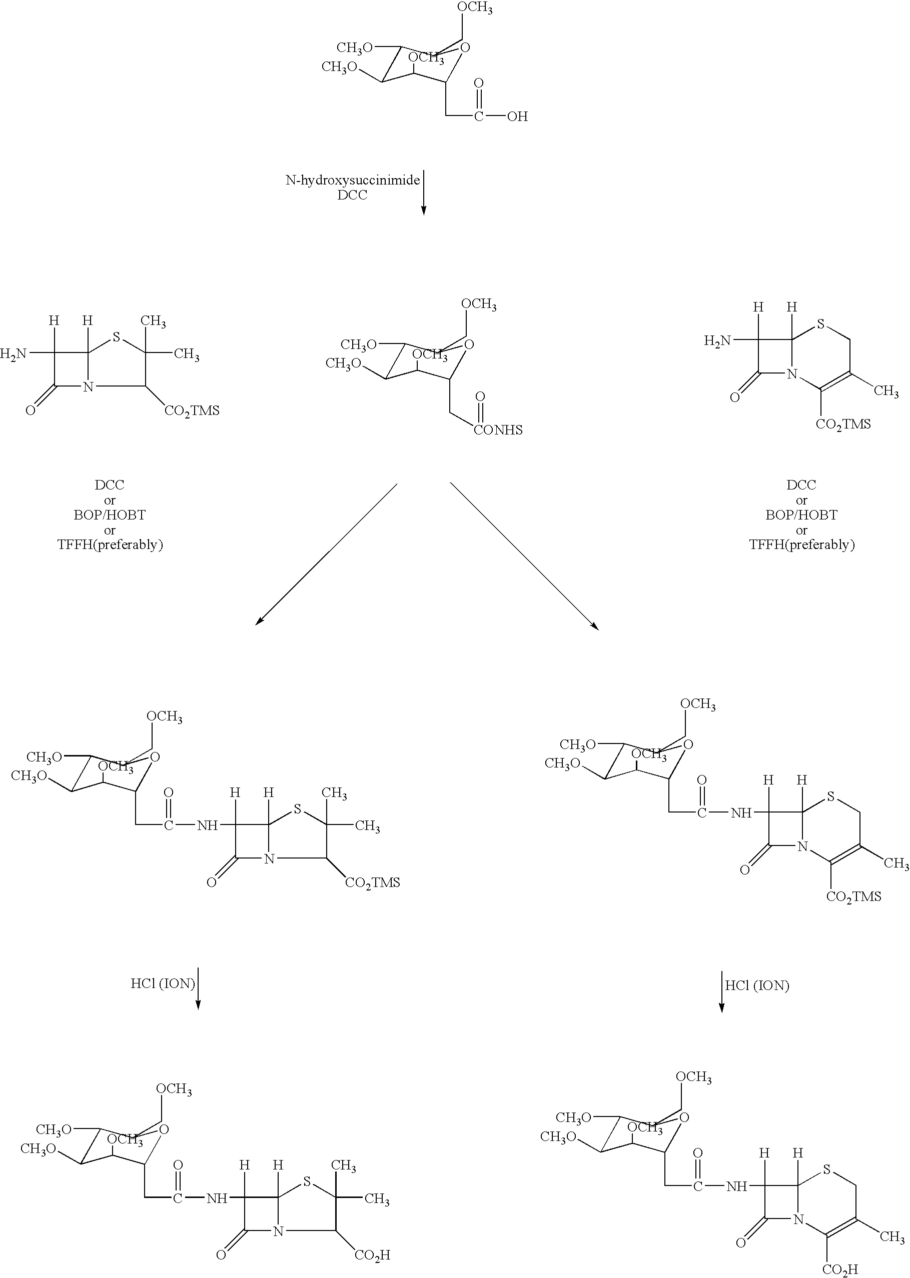

### Example 4

### Peptide and Pseudopeptide Derivatives of Mannosylacetic Acid

Following the methods disclosed above and in our co-pending U.S. provisional application Ser. No. 60/274,846 entitled STABILIZED L-Xxx-Yyy-L-L PEPTIDOMIMETICS by Amo F. Spatola and Anne-Marie Leduc, the teaching of which is hereby incorporated by reference, the following C-sugar glycopeptides have been synthesized:
1. mannosylacetyl-Leu-Glu-Gln-Leu-Leu-OH.
2. mannosylacetyl-Leu-2-Nal φ[CH₂NH]Gln-Leu-Leu-OH.

Those skilled in the art will recognize that many modifications, substitutions and extensions of the present invention disclosed herein may be readily made without departing from the teachings and scope of the appended claims. Therefore, the scope of the claims is not limited to the embodiments herein described in detail, but covers all such modifications, substitutions and extensions of said embodiments. All references cited herein are hereby incorporated by reference.

1. An alkylated C-sugar of the formula 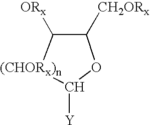
wherein n=0, 1, 2, 3 or 4,
Rₓ=aryl, alkyl, or halogen-substituted aryl or alkyl,

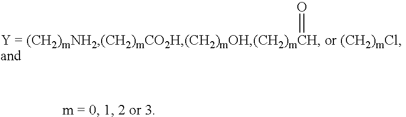

2. An alkylated C-sugar of the formula 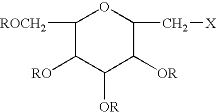
wherein X═CO₂H, CHO, OH, NH₂, Cl, Br or CH₂OH, and
R=alkyl or aryl.

3. A method of making the alkylated C-sugar according to claim 1, comprising alkylating a C-sugar to eliminate hydroxyl groups and obtain ether linkages.

4. A method of making the alkylated C-sugar according to claim 2, comprising alkylating a C-sugar to eliminate hydroxyl groups and obtain ether linkages.

5. The method according to claim 3, wherein the C-sugar to be alkylated is derived from mannose, glucose or galactose.

6. The method according to claim 4, wherein the C-sugar to be alkylated is derived from mannose, glucose or galactose.

7. The method according to claim 3, wherein the alkylated C-sugar is structured to comprise a free carboxylic acid, amine or other functional groups which facilitate coupling of said alkylated C-sugar to a drug or peptide.

8. The method according to claim 4, wherein the alkylated C-sugar is structured to comprise a free carboxylic acid, amine or other functional groups which facilitate coupling of said alkylated C-sugar to a drug or peptide.

9. A composition comprising a drug and the alkylated C-sugar according to claim 1.

10. A composition comprising a drug and the alkylated C-sugar according to claim 2.

11. The composition according to claim 9, wherein the alkylated C-sugar serves as a bioconjugate.

12. The composition according to claim 10, wherein the alkylated C-sugar serves as a bioconjugate.

13. The composition according to claim 9, wherein the drug is a peptide or polypeptide.

14. The composition according to claim 10, wherein the drug is a peptide or polypeptide.

15. The composition according to claim 9, wherein the alkylated C-sugar is coupled to said drug.

16. The composition according to claim 10, wherein the alkylated C-sugar is coupled to said drug.

17. A method of making the composition according to claim 9, comprising coupling said alkylated C-sugar to said drug.

18. A method of making the composition according to claim 10, comprising coupling said alkylated C-sugar to said drug.

19. A bioconjugate comprising the alkylated C-sugar according to claim 1.

20. A bioconjugate comprising the alkylated C-sugar according to claim 2.

21. A glycopeptide comprising the alkylated C-sugar according to claim 1, said alkylated C-sugar being coupled to the N-terminal, C-terminal and/or side chain of a peptide or polypeptide.

22. A glycopeptide comprising the alkylated C-sugar according to claim 2, said alkylated C-sugar being coupled to the N-terminal, C-terminal and/or side chain of a peptide or polypeptide.

23. A method of making the glycopeptide according to claim 21, comprising coupling said alkylated C-sugar to the N-terminal, C-terminal and/or side chain of the peptide or polypeptide.

24. A method of making the glycopeptide according to claim 22, comprising coupling said alkylated C-sugar to the N-terminal, C-terminal and/or side chain of the peptide or polypeptide.
